(19) United States
(12) Patent Application Publication
(10) Pub. No.: US 2016/0045119 A1
(43) Pub. Date: Feb. 18, 2016

(54) External Calibration and Recalibration for a Blood Pressure Monitor

(71) Applicants: Gerdt David, Faber, VA (US); Martin Baruch, Charlottesville, VA (US)

(72) Inventors: Gerdt David, Faber, VA (US); Martin Baruch, Charlottesville, VA (US)

(21) Appl. No.: 14/462,484

(22) Filed: Aug. 18, 2014

(51) Int. Cl. A61B 5/021; A61B 5/022; A61B 5/00

(52) U.S. Cl. CPC A61B 5/02108; A61B 5/6826; A61B 5/7221; A61B 5/022; A61B 5/0022

(57) ABSTRACT

A method and system for the external calibration and recalibration of a blood pressure monitor comprising a continuous pulse waveform detector, a pulse decomposition analyzer for analyzing high resolution pulse wave forms, and an absolute blood pressure monitor. Upon startup, the pulse waveform detector is initialized by the pulse decomposition analyzer based upon a reading from the absolute blood pressure monitor. Upon detection of a physiological event from the pulse decomposition analyzer, the absolute blood pressure monitor is directed to take a reading which is then used to recalibrate the pulse waveform detector.

### CROSS REFERENCE TO PRIOR APPLICATIONS

The disclosures of U.S. Pat. Nos. 6,723,054, 7,087,025, 8,100,835, Ser. No. 11/803,643, US-2007-0287923-A U.S. Ser. No. 12/537,228, Ser. Nos. 13/231,703, 12/854.954, US 2012-0238887, US-2005-0096551-A1 and US-2010_{—}0262022, PCT/US10/43914, PCT/US07/11662, U.S. Ser. No. 14/024,594, U.S. 61/946,277 and PCT/US11/47461 are incorporated herein by reference, as though recited in full.

### FIELD OF THE INVENTION

A Pulse Waveform Detection (PWD) device combined with Pulse Decomposition Analysis (PDA) constitutes a relative beat by beat blood pressure monitor.

### BACKGROUND OF THE INVENTION

It is well recognized that blood pressure measurements are an important diagnostic tool for medical professionals. Blood pressure measurements have been used to help diagnose and manage such conditions as hypertension and stress and to monitor the efficacy of drug treatments. The American Heart Association recommends home monitoring for all people with high blood pressure to assist healthcare providers.

Blood pressure may be measured invasively with an arterial catheter which has the advantage of providing highly accurate readings but which suffer from such disadvantages as potential harm to the patient, not portable, being relatively expensive, among others. There are various non-invasive methods such as the ausculatory method utilizing a manual sphygomanometer and a stethoscope, photoplethysmographs (PPGs), and piezoelectric sensors.

The ausculatory method involves wrapping an inflatable cuff typically around the upper arm of a patient, inflating the cuff to a pressure sufficient to trap the brachial artery against the bone so that the blood pressure pulse in the brachial artery is communicated to the surface where a stethoscope may be used to listen for Korkotoff sounds. Readings are taken in a manner well known in the art. The advantage of the ausculatory method is that the reading is an absolute blood pressure measurement. The disadvantages of the ausculatory method include discomfort to the patient, partial occlusion of the artery being measured, 1-2 skilled operators are required, and care should be taken as to how many times the method is applied in succession because of the arterial occlusion. The ausculatory method is considered the gold standard for office use.

However, it is often desirable to have a patient have many measurements over a period of hours, days, or even longer. Accordingly, it also desirable to be able to take these readings automatically. The comfort of the patient is also desirable when the Blood Pressure Monitor (BPM) is to be worn over a long period of time including during sleep. The Ausculatory Method is undesriable because of the expense of providing trained staff for all the readings and also because of the discomfort caused by the cuff when inflated.

Presently, an automatic hydrostatic device is used which is a relative beat by beat BPM. The phrase, “beat by beat” is used to distinguish a hydrostatic cuff diagnostic device from continuous blood pressure monitors that can be programmed to take pressure values every ten minutes or even every hour. In these systems, nothing is known about the values in between programmed intervals.

### BRIEF DESCRIPTION OF THE DRAWINGS

FIG. 1 is a dorsal perspective view of the finger cuff on a user's hand and receiving device in accordance with the present invention; and

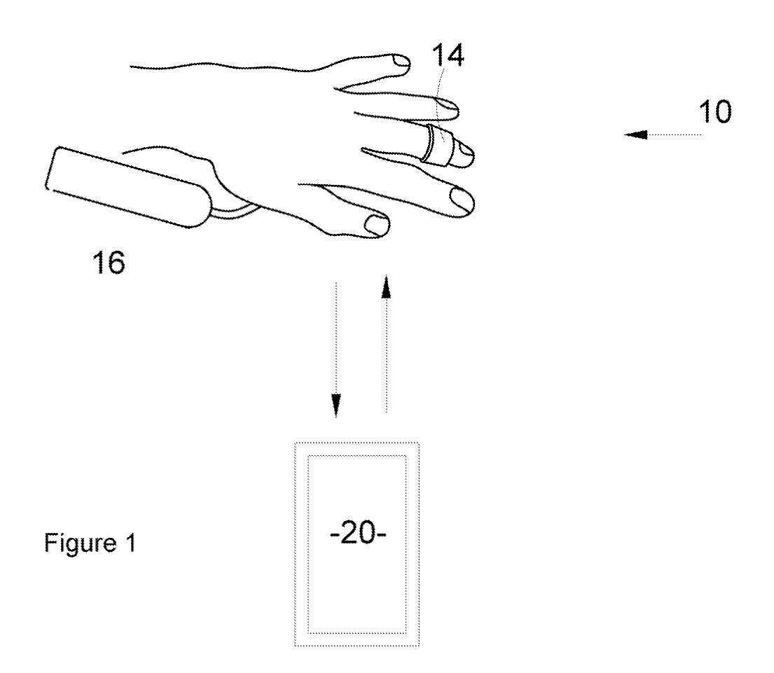

FIG. 2 is a functional representation of a system of the present invention

### SUMMARY OF THE INVENTION

An object of the present invention is to avoid the problems and disadvantages of blood pressure monitors which can not automatically recalibrate.

In one embodiment of the present invention, the Blood Pressure Monitor comprises a pulse waveform detector (PWD) capable of being worn by a patient which is further capable of detecting said patient's pulse waveform, a control device having means of communicating with the PWD, means of communication with an Absolute Blood Pressure Monitor, and having Pulse Decomposition Analysis (PDA) means. The Control Device calibrates the PWD according to information from the Absolute Blood Pressure Monitor. The Control Device receives high resolution pulse waveforms from the PWD. The Control Device sends a calibration signal to the PWD based on information from the ABPM when a Recalibration Event occurs. A Recalibration Event occurs either at a predetermined time or one or more predetermined physiological events are detected. Physiological events are detected by the PDA analyzing the pulse waveforms received from the PWD in a manner disclosed in the referenced copending applications and patents including U.S. Pat. Nos. 7,087,025 and 8,100,835.

In another embodiment the Pulse Waveform Detector is the finger cuff disclosed in U.S. patent application Ser. No. 14/024,594

In another embodiment is a method of calibrating and recalibrating the pulse waveform detector comprising sensing predetermined physiological parameters, activating an external absolute blood pressure system and determining a recalibrate factor, communicating said recalibrate factor to said pulse waveform detector.

While the present invention is primarily directed toward human beings, the principles taught herein will also have veterinary application with animals. Valuable race horses, for example, may warrant the use of and would benefit from the system described herein.

### DETAILED DESCRIPTION OF THE INVENTION

### Definitions

As used herein the term “about” refers to a variation of +/−15%.

As used herein the term “beat by beat” shall refer to a hydrostatic cuff diagnostic device from continuous blood pressure monitors.

As used herein the term “cuff” shall refer to wrap, cuff or other covering that maintains a predetermined contact pressure for use on a phalange to continuously monitor blood pressure.

As used herein the term “recording device” shall refer to a microprocessor having receiving and transmission capabilities to receive and transmit data from the hydrostatic cuff.

As used herein the term “receiving device” shall refer to any device having a microprocessor capable receiving and calculating data from a hydrostatic cuff recording device.

As used herein the term “hydrostatic cuff diagnostic system” shall refer to a diagnostic device comprising a cuff, recording device and receiving device as a single or multiple unit.

As used herein the term “PDA” shall refer to a pulse decomposition analysis as disclosed in U.S. Pat. Nos. 6,723,054, 7,087,025, 8,100,835, Ser. No. 11/803,643, US-2007-0287923-A U.S. Ser. No. 12/537,228, Ser. Nos. 13/231,703, 12/854,954, US 2012-0238887, US-2005-0096551-A1 and US-2010_{—}0262022, PCT/US10/43914, PCT/US07/11662, and PCT/US11/47461.

A pulse waveform detector combined with Pulse Decomposition Analysis (PDA) constitutes a relative beat by beat blood pressure monitor suitable, with external calibration, for replacing most arterial catheters that do not require blood gas sampling (10-20%). Because the pulse waveform detector is relative, it requires an initialization and it is necessary to measure the absolute blood pressure, which can be most easily accomplished by a manual or automatic sphygmomanometer. These absolute values are then input into the PDA algorithm, which initializes a hydrostatic cuff diagnostic device as an accurate relative blood pressure monitor that reports beat by beat blood pressure to within the guidelines established by FDA, or other predetermined guidelines, namely, ANSI/AAMI SP10:2002. The pulse waveform detector need only be able to capture and transmit a high resolution pulse waveform as taught in copending application U.S. Ser. No. 14/024,594 wherein the pulse waveform detector is the hydrostatic finger cuff. A hydrostatic cuff is the preferred embodiment's pulse waveform detector although any pulse waveform detector which can, for example, capture a pulse waveform with sufficient resolution to identify pulsus paradoxus will suffice. Calibrating a hydrostatic cuff comprises inflating the cuff to a pressure sufficient to report beat by beat blood pressure within the guidelines established by the FDA as referenced herein. Because it is an element of the preferred embodiment, hydrostatic cuff diagnostic device is used interchangably with pulse waveform detector. In manual methods, an ordinary manual upper arm cuff and a stethoscope can be used to arrive at the absolute blood pressure and these values can then be manually input into the PDA software thereby effecting the initialization or recalibration.

Like other relative beat by beat blood pressure monitors, a hydrostatic cuff diagnostic device loses calibration over time. The hydrostatic cuff diagnostic device has demonstrated that it remain calibrated to FDA guidelines for 40 minutes in the. In emergency medicine, a hydrostatic cuff diagnostic device can require recalibration more frequently. For some patients or home monitors, calibration may last for days or weeks, but hydrostatic cuff diagnostic device will need to be recalibrated periodically, however no matter the time period the hydrostatic cuff diagnostic system 10 will need to be recalibrated periodically.

In addition to time and predetermined usage, two physiological parameters are known that will cause hydrostatic cuff diagnostic device/PDA to lose calibration regardless of the frequency of recalibration. These are a change in heart rate by more than about 20%-30% or if the arterial stiffness changes by more than about 10%. If either of these events occur, a recalibration is necessary for the hydrostatic cuff diagnostic system 10 to remain within FDA guidelines. Additionally, it may be desirable to recalibrate the BPM during a great many physiological conditions such as hypovolemia, hemorrhaging, pulsus paradoxus, stress, heart patters such as arrhythmia, temperature, respiration, and some mental conditions.

In manual recalibration methods, an ordinary manual upper arm cuff and a stethoscope can be used to arrive at the absolute blood pressure and these values can then be manually input into the PDA software thereby effecting the initialization or recalibration. Manual methods, however require personnel time which, especially in busy hospitals, in field hospitals, emergency facilities, etc.,

In automatic time based methods, the PDA program in the receiving device knows when to recalibrate based upon a predetermine and preprogrammed time period, with recalibration being generally in the range from 5 to 240 minutes, and preferably about 40 minutes. When recalibration is necessary, the system PDA program contacts an automatic sphygmomanometer that inflates a cuff on the patients arm. The data received from the sphygmomanometer is transmitted to the hydrostatic cuff recording device 16 and/or receiving device 20, providing the updated absolute numbers.

Alternatively, or additionally, recalibration can be automatically called for by the hydrostatic cuff diagnostic device measuring changes in arterial stiffness, or when large heart rate changes, in the range from 20% and larger, are sensed. The result is that the hydrostatic cuff diagnostic device/PDA essentially knows when a calibration is needed. Other physiological parameters or states that are sensed, such as hypovolemia or blood volume changes or heart rate patterns such as arrhythmia, or temperature or respiration rate changes, could cause recalibration, and these physiological parameters or states are determined either by the hydrostatic cuff diagnostic device/PDA or by another external physiological sensor. Thus, the hydrostatic cuff diagnostic device could potentially call for a recalibration due to the observation of any physiological parameter as taught in the reference co-pending applications and patents.

The recalibration called for by the hydrostatic cuff diagnostic device can be communicated to the automatic cuff by a wire or by wireless technologies such as Bluetooth. In the Bluetooth method, hydrostatic cuff diagnostic device sends a command to the Bluetooth enabled absolute blood pressure sphygmomanometer that causes the absolute system to inflate and to measure the desired parameters, such as a blood pressure of 120/80 mmHg, for instance. The automatic cuff then radios these values back to the hydrostatic cuff diagnostic device and they are used to recalibrate the hydrostatic cuff diagnostic device so that the hydrostatic cuff diagnostic device's measured beat by beat blood pressures are within FDA guidelines.

The finger cuff disclosed in U.S. patent application Ser. No. 14/024,594 provides many benefits including, wearer comfort, little to no arterial occlusion, and it is sufficiently low cost that they may be disposed of which can improve hygiene by simply replacing when soiled or contaminated.

The recalibration called for by the recording device 16 can be communicated to the receiving device 20 by a wire or by wireless technologies such as Bluetooth. In the Bluetooth method, receiving device 20 sends a command to the Bluetooth enabled absolute blood pressure sphygmomanometer that causes the absolute system to inflate and to measure the desired parameters, such as 120/80 mmHg for instance. The automatic cuff then radios these values back to the hydrostatic cuff diagnostic device and they are used to recalibrate the hydrostatic cuff diagnostic device so that the hydrostatic cuff diagnostic device's measured beat by beat blood pressures are within FDA guidelines.

The raw data received from the recording device 16 and sent to the receiving device 20 is translated via an algorithm that monitors the patient's normal, or desired, pressure as well as determines recalibration. In some embodiments, the raw data can be translated directly within the recording device. The monitoring algorithm is:

1 Pa=145×10⁻⁶ psi 1kPa=1000 Pa=145×10⁻³ psi.

1 psi=6.89 kPa 40 mm Hg=40×133.3 Pa=5.33 kPa Pulse Decomposition Algorithm

The basic components of the algorithm are 1—a peak finder that identifies heartbeats in the derivative data stream, 2—a differentiator that produces the second derivative of the detected heart beat which is then used to find the inversions corresponding to the locations of the component pulses, 3—a digital integrator, implemented as a Bessel filter, that generates the integrated pulse wave form from the differentiated raw signal stream, and from which relative component pulse amplitudes are determined and 4—a low-pass filter that enables identification of the primary systolic peak. Furthermore the frequency content of the data stream is continuously analyzed in order to calculate signal to noise (S/N) figures of merit that determine whether signal fidelity is sufficiently high to permit peak detection and analysis.

Once the temporal locations of the reflection component pulses and the systolic peak are identified, the T13 interval, the time delay between systolic (P1) and iliac peak (P3), is calculated. The P2P1 ratio is calculated using the amplitudes of the P2 peak and the systolic peak, in the integrated pulse spectrum.

Although the drawing herein illustrates a finger cuff, it should be noted that the cuff can be used on any phalange having size to enable contact with the cuff, although best results have been found to be on the first phalange of the thumb.

In situations where there are multiple patients being monitored, each hydrostatic cuff recording device 16 can be assigned a different frequency and a single recording device 20 used to monitor multiple patients.

In some embodiments it can be advantageous to embed the recording device 20 in the cuff. Although this is a more expensive alternative, in some situations having a small, compact unit is preferable.

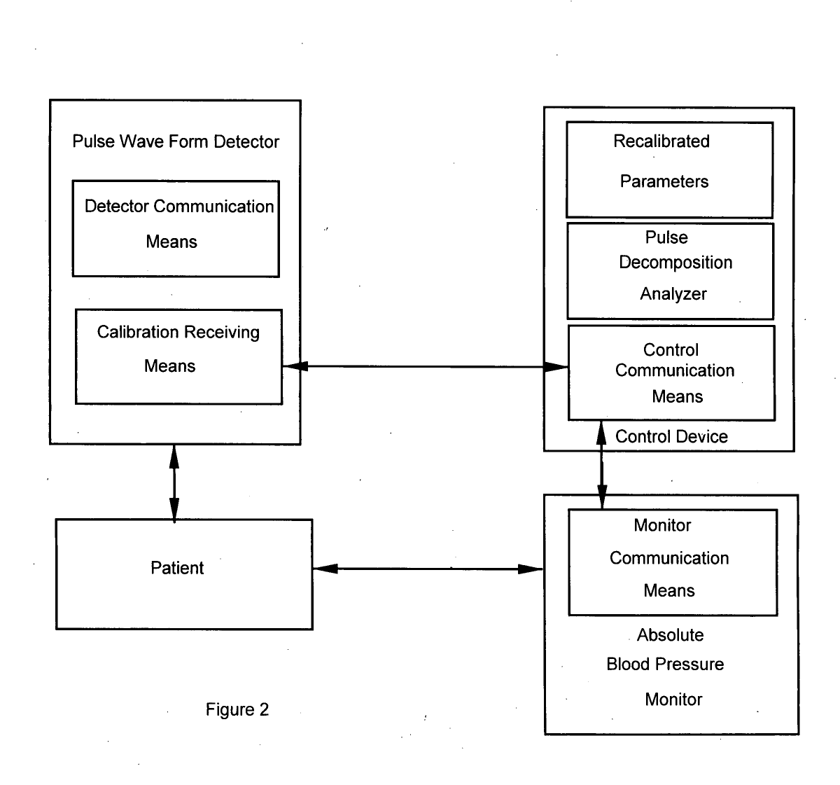

While FIG. 2 shows a functional representation of an embodiment of a system of the present invention, it should not be read as a limitation. For example, the Absolute Blood Pressure Monitor is shown as having a monitor communication means. However, the Absolute Blood Pressure Monitor may comprise a manual sphygmomanometer and stethoscope which may be used to take a reading and then enter that reading into the control device. The control device might be implemented on a general purpose computer in whole with the pulse decomposition analyzer being implemented in software or in part where the computer is in communication with hardware implemented pulse decomposition analyzer. There is no reason why the control device could not be co-located with the pulse waveform detector but control device size and signal noise would need to be accounted for.

While illustrative embodiments of the invention have been described herein, the present invention is not limited to the various preferred embodiments described herein, but includes an and all embodiments having equivalent elements, modifications, omissions, combinations (e.g., of aspects across various embodiments), adaptations and/or alterations as would be appreciated by those in the art based on the present disclosure. The limitations in the claims are to be interpreted broadly based on the language employed in the claims and not limited to examples described in the present specification or during the prosecution of the application, which examples are to be construed as non-exclusive. For example, in the present disclosure, the term “preferably” is non-exclusive and means “preferably, but not limited to.”

What is claimed is:

1. A method of calibrating and recalibrating a hydrostatic cuff diagnostic device for detecting a pulse waveform comprising, sensing a physiological parameters sensed changes the calibration of the hydrostatic cuff diagnostic device,
activating an external absolute blood pressure system and determining a recalibrate factor,
communicating said recalibration factor to said diagnostic device,
recalibrating said diagnostic device using said recalibration factor,
wherein said recalibration factor is based on measured changes in physiological parameters.

2. The method of claim 1, wherein said hydrostatic cuff diagnostic device is a finger cuff.

3. The method of claim 1, wherein said communicating of said recalibration factor is by means of a wireless signal.

4. The method of claim 1, further comprising said communication of said recalibration factor is by a radio enabled automatic external blood pressure measurement system and wherein a resulting absolute blood pressure is communicated to said hydrostatic cuff diagnostic device via radio.

5. A blood pressure monitor comprising:
a control device having a pulse decomposition analyzer, recalibration parameters, a control communication means; and
a pulse waveform detector capable of being worn by an animate being and further being capable of continually detecting said animate being's pulse waveform, and having detector communication means for continually communicating said pulse wave form and means for receiving calibration instructions;
an absolute blood pressure monitor for detecting the absolute blood pressure of said animate being and monitor communication means capable of communicating said absolute blood pressure to said control device and for receiving instructions to initiate said detection of said absolute blood pressure of said animate being;
wherein said control communication means is capable of communicating with said detector communication means and said monitor communication means, and wherein said control device continually analyzes said pulse waveform with said pulse decomposition analyzer and compares said analysis against said recalibration parameters and initiates a calibration process;
wherein said calibration process comprises:
sending an absolute blood pressure request to said absolute blood pressure monitor to measure the absolute blood pressure;
communicating the absolute blood pressure to said control device;
generating calibration instructions based upon said absolute blood pressure; and
communicating calibration instructions to said pulse waveform detector, and
wherein said control device also initiates said calibration process upon startup of said blood pressure monitor.

6. The system of claim 5 wherein said pulse waveform detector is a finger cuff.

7. The system of claim 5 wherein
said control device is implemented on a general purpose computer;
said pulse decomposition analyzer is a software component on said general purpose computer.

8. The system of claim 7 wherein at least one of said detector communication means and said monitor communication means comprises Bluetooth technology.

9. The system of claim 7 wherein at least one of said detector communication means and said monitor communication means comprises a cable using USB technology.
